SEQUENCE LISTING (9 sequences)

SEQ ID NO: 1 - DNA, 28 bp, Artificial Sequence
  misc_feature (1)..(26): The nucleotide sequence of a selected target fragment in the first exon region of Sp3 and Sp4 in the S-RNase gene as a self-incompatibility determining gene. n=a or g or c or t or void; and the number of the existing “n” is from 16 to 26.
nnnnnnnnnnnnnnnnnnnnnnnnnngg

SEQ ID NO: 2 - DNA, 726 bp, Artificial Sequence
  Sp3 nucleotide sequence
atggttaaatcacaactcacagcagctctcttcattgtgctttttgctctttctcccgcttatggggatttcgattccctccaactggtattaacatggccagcatcattttgccatgttaatgattgtgtgcgaatagctccaaaaaacttcacgattcacgggctttggccggataaagagggaacggtgctgcagaactgcaagccaaaacctaagtatgttaatttcaaggtaagcaatagcattttttttagagcccgcttttccgttcagttcaatttacttgaaagattcttttcgaaatgcttacaggataagatgttcaacgatcttgacaaacactggattcagttgaagtttgatgaagattatggtgaaaaggaacaacctttatggctctatcaatatttaaagcatggatcttgttgtcagaaaatgtacaaccaaaacacgtattttagtctagccttgcgcttaaaagacaggtttgatcttctgagaactctccaaatacatcatatttttcctggatcaagttatacatttaagaaaatctttgatgccgtcaagacagctactcaaatggatcctgaccttaagtgtactaaaggagcaccggaattatatgaaataggcatatgtttcaccccaaatgcagatgctctgattccttgtcgtcaaagtaatacatgcgataggacgggaaaaatcttttttcgctga

SEQ ID NO: 3 - DNA, 789 bp, Artificial Sequence
  Sp4 nucleotide sequence
atggctaaatcgcagcttgtgtcagcacttttcgttttctttttttctctttctcccatttatggggatttcgactatatgcaacttgtcttaacatggccaagatctttttgttaccctagaggattctgcaacagaatacctccgaacaacttcacgattcacgggctttggccggataagaagcctatgcgcggccagttacaattctgcacttctgatgactatataaaatttactccggtaagttacagcattatttacttcagccaaatagtcttaattttttatttttcatttgaatagcttgcatttcctttctgttaatttttagtagaaatcttttacaaatttccacagggtagtgtactcgatgctctggaccaccactggattcagttgaagtttgaacgcgaaataggaatccgtgatcaacctctctggaaagatcaatacaaaaagcatggaacatgttgtttaccgcgctacaatcagttgcagtattttttactagccatgcgactgaaagaaaagtttgatcttttgacaactctcagaacccatggaattactcctggtacaaaacatacatttaaaaaaatccaagatgccatcaagacagtcactcaagaggttcctgacctcaagtgcgttgaaaatattcaaggcgtattggaactatacgagataggaatatgttttactcccgaggcagatagtctatttccttgtcgtcaaagtaaatcatgccacccaacagaaaacccgctgattttgtttcgattatga

SEQ ID NO: 4 - DNA, 23 bp, Artificial Sequence
  misc_feature (1)..(4): The forward oligonucleotide strand synthesized at the target site.  n=a or t or c or g, and positions 1 to 4 constitute a cohesive end for ligation.
nnnnacgattcacgggctttggc

SEQ ID NO: 5 - DNA, 23 bp, Artificial Sequence
  misc_feature (1)..(4): The reverse oligonucleotide strand complementary to the forward oligonucleotide strand at the target site.  n=a or t or c or g, and positions 1 to 4 constitute a cohesive end for ligation.
nnnngccaaagcccgtgaatcgt

SEQ ID NO: 6 - DNA, 20 bp, Artificial Sequence
  SP3 amplification-specific forward primers
ggggaaactggaaaatggtt

SEQ ID NO: 7 - DNA, 21 bp, Artificial Sequence
  SP3 amplification-specific reverse primers
atgtgaagttgttcagcgaaa

SEQ ID NO: 8 - DNA, 22 bp, Artificial Sequence
  SP4 amplification-specific forward primers
caacaaaatggctaaatcgcag

SEQ ID NO: 9 - DNA, 20 bp, Artificial Sequence
  SP4 amplification-specific reverse primers
ggttttctgttgggtggcat

(19) United States
(12) Patent Application Publication
(10) Pub. No.: US 2021/0054391 A1
(43) Pub. Date: Feb. 25, 2021

(54) METHOD FOR OVERCOMING SELF-INCOMPATIBILITY OF DIPLOID POTATOES

(71) Applicants: Agricultural Genomics Institute, Chinese Academy of Agricultural Sciences, Shenzhen (CN); Agricultural Genomics Institute at Shenzhen, Chinese Academy of Agricultural Sciences, Shenzhen (CN)

(72) Inventors: Sanwen HUANG, Shenzhen (CN); Chunzhi ZHANG, Shenzhen (CN); Zhen PENG, Shenzhen (CN); Mingwang YE, Shenzhen (CN)

(21) Appl. No.: 16/960,292

(22) PCT Filed: Apr. 4, 2019
(86) PCT No.: PCT/CN2019/081515

§ 371 Date: Jul. 6, 2020

(30) Foreign Application Priority Data
Apr. 8, 2018 (CN) 201810308517.5
Jan. 18, 2019 (CN) 201910108556.5

(51) Int. Cl. C12N 15/82

(52) U.S. Cl. CPC C12N 15/8213

(57) ABSTRACT

Disclosed is a method for overcoming self-incompatibility of diploid potatoes, including: (1) selecting a target fragment; (2) constructing a gene-targeting recombinant vector; (3) achieving a loss-of-function mutation of the intracellular S-RNase gene; (4) regenerating a plurality of potato plants; (5) specifically amplifying a DNA segment containing the target fragment of the S-RNase gene in a regenerated plant; (6) selecting a regenerated plant in which the S-RNase gene is edited; (7) further screening the selected gene-edited plant for a diploid gene-edited plant line; (8) propagating and planting the selected gene-edited plant line, and identifying the self-compatible phenotype at the flowering stage; and (9) sequencing the gene amplification products of the harvested offspring of the self-compatible plant, and detecting the inheritance and isolation of the offspring in which the target gene is edited. The invention provides a simple, accurate and efficient method for overcoming the self-incompatibility of diploid potatoes.

### TECHNICAL FIELD

The invention belongs to the technical field of biotechnology and genetic breeding, particularly to a method for overcoming self-incompatibility of diploid potatoes.

### BACKGROUND

A diploid potato belongs to gametophytic self-incompatibility type, and its pollen tubes can germinate on the stigma and elongate into the style, but the growth is subsequently inhibited. This trait is controlled by the S-RNase gene, and the expression of this gene inhibits the elongation of the pollen tubes, making it difficult for the potatoes to obtain an inbred plant line.

Previous studies have found that a tobacco S-RNase gene is only slightly expressed during the period from flower bud to bud stage, while higher S-RNase protein enrichment is detected in flowering stage. Our research on potato pistil proteins has also reaches the similar conclusion, and this creates the possibility of overcoming self-incompatibility of potatoes through self-crossing at the bud stage. However, self-crossing at the bud stage has higher requirements for pollination times and environment, lower fruit setting rate and fewer seeds, and the plants growing from the seeds of the self-crossing are still self-incompatible plants. The method is time-consuming and laborious, and the cost is extremely high; the self-compatibility is only exhibited in the generation for self-crossing at the bud stage, and is not heritable. The method of self-crossing at the bud stage cannot achieve the creation of self-compatible materials.

In 1998, Hosaka and Hanneman mapped a Sli (S-locus inhibitor) gene locus derived from the wild species S. chacoense, this gene confers self-compatibility to diploid potatoes. However, the infiltration of the Sli gene into a cultivar potato will inevitably bring in unfavorable traits, such as longer stolons, smaller potato tubers, and increased steroidal glycosides and alkaloids.

Therefore, it is desirable to create self-compatible potato materials in a better way. At present, no research institution has made breakthroughs in this regard.

### SUMMARY

In order to solve the problem that there is no better method to create a self-compatible potato material in the prior art, the present invention provides a method for overcoming self-incompatibility of diploid potatoes. The purpose of the invention is to find a simple, accurate and efficient method to overcome self-incompatibility of diploid potatoes, i.e., overcoming the self-incompatibility barrier of diploid potatoes, thereby providing core technology support for the creation of a self-compatible material and a homozygous inbred plant line.

In order to achieve the above object, the present invention provides the following technical solutions. The present invention provides a method for overcoming self-incompatibility of diploid potatoes, which comprises the following steps:

(1) selecting a target fragment in the gene regions of Sₚ₃ and Sₚ₄ in the S-RNase gene as a potato self-incompatibility determining gene;

(2) constructing a CRISPR/Cas9 recombinant vector for diploid potato S-RNase gene-targeting according to the nucleic acid sequence of the target fragment obtained in step (1);

(3) introducing the recombinant vector obtained in the step (2) into potato cells, inducing the co-expression of the guide RNA expression cassette of the target fragment and the Cas9 nuclease expression cassette in the cell, cleaving the double-stranded target fragment of the S-RNase gene to trigger the DNA repair function of the potato cell itself, and causing random insertion or deletion of bases at the target site, thereby achieving a loss-of-function mutation of the intracellular S-RNase gene;

(4) regenerating a plurality of potato plants from the potato cells introduced with the recombinant vector, and screening the marker gene in the selected regeneration plants;

(5) specifically amplifying a DNA segment with the target fragment in the S-RNase gene of the selected regeneration plants by genomic PCR method, and sequencing the amplified products;

(6) selecting a regenerated plant in which the S-RNase gene is edited;

(7) detecting the ploidy of the selected gene-edited plant to select a diploid gene-edited plant line;

(8) propagating and planting the selected gene-edited plant line, and identifying the self-compatible phenotype at the flowering stage; and

(9) harvesting the seeds of the self-compatible plant line, extracting the genomic DNA of the offspring, and specifically amplifying a DNA segment with the target fragment in the S-RNase gene of the selected offspring by PCR method, then sequencing the amplified products and detecting the inheritance and isolation of the edited target gene in the offspring.

The gene regions of Sₚ₃ and Sₚ₄ in the S-RNase gene as a potato self-incompatibility determining gene comprise an exon and a promoter, or a portion thereof.

Further, in the step (1) the target fragment is located on the target gene S-RNase, and one strand of the target fragment has the nucleic acid sequence structure as shown in SEQ ID No:1. For example, wherein the target fragment is located on the target S-RNase gene, one strand of the target fragment has a 5′-(N)_{X}-NGG-3′ structure, and (N)_{X} represents a base sequence having the base number of X {N₁, N₂, . . . , N_{X}}, and each of N₁, N₂, . . . N_{X} represents any one of bases A, G, C, and T, and N in NGG is any one of A, G, C, and T. X is an integer from 15 to 25, preferably, said X is an integer from 17 to 23; more preferably, said X is 18, 19, 20, or 21.

Further, in the step (2) the recombinant vector comprises the target fragment, wherein the target fragment is the nucleic acid sequence of SEQ ID No:1 or a sequence complementary thereof.

Another aspect of the present invention provides a potato plant, and a plant part, a tuber or tuber part, a plant cell, a pollen or a seed thereof, it comprises a loss-of-function mutation of the S-RNase gene, wherein the nucleotide sequence of the S-RNase gene is the sequence shown in SEQ ID NO:2 (Sₚ₃), or a complementary sequence, a degenerate sequence, or a homologous sequence thereof; and/or the sequence shown in SEQ ID NO:3 (Sₚ₄), or a complement sequence, a degenerate sequence, or a homologous sequence thereof.

In a particular embodiment of the present invention, the homologous sequence of the nucleotide sequence of the S-RNase protein may be a polynucleotide hybridizing with a nucleotide sequence in SEQ ID NO:2 and/or SEQ ID NO:3 or a complementary sequence thereof under stringent conditions, or a fragment of the polynucleotide, wherein such a polynucleotide or a fragment thereof does not express the S-RNase protein.

The “stringent conditions” described herein may be any kind of the followings: low stringency conditions, medium stringency conditions, and high stringency conditions, preferably high stringency conditions. Exemplarily, the “low stringency conditions” may be the conditions of 30° C., 5×SSC, 5×Denhardts solution, 0.5% SDS, 52% formamide; “medium stringent conditions” may be the conditions of 40° C., 5×SSC, 5×Denhardts solution, 0.5% SDS, 52% formamide; “high stringency conditions” may be the conditions of 50° C., 5×SSC, 5×Denhardts solution, 0.5% SDS, 52% formamide. Those skilled in the art will appreciate that the higher the temperature, the more highly homologous polynucleotides can be obtained. In addition, one skilled in the art can select a comprehensive result produced by a plurality of factors affecting the stringency of hybridization such as the temperature, probe concentration, probe length, ionic strength, time, and salt concentration, etc. so as to achieve a corresponding stringency.

In addition, the hybridizable polynucleotide may be such a polynucleotide, when calculated by a homology search software such as FASTA or BLAST with default parameters set by the system, the polynucleotide has about 30% or more, 40% or more, 50 or more, 60% or more, about 70% or more, 71% or more, 72% or more, 73% or more, 74% or more, 75% or more, 76% or more, 77% or more, 78% or more, 79% or more, 80% or more, 81% or more, 82% or more, 83% or more, 84% or more, 85% or more, 86% or more, 87% or more, 88% or more, 89% or more, 90% or more, 91% or more, 92% or more, 93% or more, 94% or more, 95% or more, 96% or more, 97% or more, 98% or more, 99% or more, 99.1% or more, 99.2% or more, 99.3% or more, 99.4% or more, 99.5% or more, 99.6% or more, 99.7% or more, 99.8% or more, or 99.9% or more homology with the polynucleotide according to the present invention.

For homology of nucleotide sequences, it may be determined by the BLAST algorithmic rules of Karlin and Altschul (Proc. Natl. Acad. Sci. USA 87: 2264-2268, 1990; Proc. Natl. Acad. Sci. USA 90: 5873, 1993). The programs based on the BLAST algorithmic rules such as BLASTN and BLASTX have been developed (Altschul S F, et al: J Mol Biol 215: 403, 1990). When analyzing the base sequence by using BLASTN, for example, the parameters are: score=100, wordlength=12; when using the BLAST and Gapped BLAST programs, the default parameter values may be set in the system using each of the programs.

In a particular embodiment of the present invention, the loss-of-function of the S-RNase gene is achieved by addition and/or deletion of (one or more) nucleotides in the gene expressing the S-RNase protein. For example, the addition of one, two or more nucleotides; or the deletion of one, two or more nucleotides; or the replacement of one, two or more nucleotides in the gene expressing the S-RNase protein.

Exemplarily, the nucleotide sequence of the S-RNase protein is the sequence shown in SEQ ID NO:2 (Sₚ₃), or a complementary sequence, a degenerate sequence, or a homologous sequence thereof; and/or the sequence shown in SEQ ID NO:3 (Sₚ₄), or a complement sequence, a degenerate sequence, or a homologous sequence thereof.

In a particular embodiment of the invention, the loss of function of the S-RNase gene is achieved by addition, deletion or replacement of (one or more) nucleotides in the sequence of ACGATTCACGGGCTTTGGCC or a complementary sequence thereof. For example, the addition of 1 to 5 nucleotides, deletion of 1 to 10 nucleotides, or replacement of 1 to 8 nucleotides, etc.

In a particular embodiment of the invention, the addition, deletion or replacement of nucleotides is achieved by a CRISPR/Cas9 recombinant vector.

In a particular embodiment of the present invention, the CRISPR/Cas9 recombinant vector is capable of targeting the gene of the S-RNase protein, wherein the nucleotide sequence of the S-RNase protein is the sequence shown in SEQ ID NO:2 (Sₚ₃), or a complementary sequence, a degenerate sequence, or a homologous sequence thereof; and/or the sequence shown in SEQ ID NO:3 (Sₚ₄), or a complement sequence, a degenerate sequence, or a homologous sequence thereof.

In a particular embodiment of the invention, the CRISPR/Cas9 recombinant vector is capable of targeting the first exon region of the S-RNase gene.

In a particular embodiment of the invention, the CRISPR/Cas9 recombinant vector is capable of targeting the sequence of ACGATTCACGGGCTTTTGGCCGG in the S-RNase gene or a complementary sequence thereof.

In a particular embodiment of the invention, the nucleotide sequence of the sgRNA in the CRISPR/Cas9 recombinant vector is:

S-RNase P3 (i.e., Seq ID No: 4):
xxxxACGATTCACGGGCTTTGGC,
S-RNase P4 (i.e., Seq ID No: 5):
xxxxGCCAAAGCCCGTGAATCGT;

wherein the portion not underlined is a sequence in above target site with deletion of NGG or a complementary sequence thereof, and the underlined portion is a cohesive end for ligation of the vector.

In another aspect, the present invention provides a CRISPR/Cas9 recombinant vector for targeted knockout of S-RNase gene, the nucleotide sequence of the S-RNase gene targeted by the CRISPR/Cas9 recombinant vector is the sequence shown in SEQ ID NO:2 (Sₚ₃), or a complementary sequence, a degenerate sequence, or a homologous sequence thereof and/or the sequence shown in SEQ ID NO:3 (Sₚ₄), or a complement sequence, a degenerate sequence, or a homologous sequence thereof.

In a particular embodiment of the invention, the CRISPR/Cas9 recombinant vector is capable of targeting the first exon region of the S-RNase gene.

In a particular embodiment of the invention, the CRISPR/Cas9 recombinant vector is capable of targeting the sequence of ACGATTCACGGGCTTTTGGCCGG in the S-RNase gene or a complementary sequence thereof.

In a particular embodiment of the invention, the nucleotide sequence of the sgRNA in the CRISPR/Cas9 recombinant vector is:

S-RNase P3 (i.e., Seq ID No: 4):
xxxxACGATTCACGGGCTTTGGC,
S-RNase P4 (i.e., Seq ID No: 5):
xxxxGCCAAAGCCCGTGAATCGT;

wherein the portion not underlined is a sequence in above target site with deletion of NGG or a complementary sequence thereof, and the underlined portion is a cohesive end for ligation of the vector.

In a particular embodiment of the invention, the construction of the CRISPR/Cas9 recombinant vector comprises the following steps:

(1) designing primers S-RNase P3 and S-RNase P4 according to the target sequence;

(2) making S-RNase P3 and S-RNase P4 to form a double-stranded DNA having cohesive ends as an insert fragment for constructing the recombinant vector;

(3) digesting the pKSE401 vector with BsaI endonuclease as a skeleton fragment of the framework recombinant vector;

(4) ligating the recombinant vector backbone fragment and the insert fragment by T4 ligase, then transferring into E. coli to screen for the CRISPR/Cas9 recombinant vector.

Exemplarily, the sequences of the primers S-RNase P3 and S-RNase P4 are as follows:

S-RNase P3 (i.e., Seq ID No: 4):
xxxxACGATTCACGGGCTTTGGC,
S-RNase P4 (i.e., Seq ID No: 5):
xxxxGCCAAAGCCCGTGAATCGT;

wherein the portion not underlined is a sequence in above target site with deletion of NGG or a complementary sequence thereof, and the underlined portion is a cohesive end for ligation of the vector.

Another aspect of the invention provides use of the above CRISPR/Cas9 recombinant vector in the preparation of knockout of S-RNase protein gene.

Another aspect of the present invention provides a method for breeding a self-compatible potato, which comprises the step of making the S-RNase gene in a potato unexpressed or inactivated, wherein the S-RNase gene is the sequence shown in SEQ ID NO:2 (Sₚ₃), or a complementary sequence, a degenerate sequence, or a homologous sequence thereof; and/or the sequence shown in SEQ ID NO:3 (Sₚ₄), or a complement sequence, a degenerate sequence, or a homologous sequence thereof.

In a particular embodiment of the present invention, the breeding method specifically includes:

(1) constructing a CRISPR/Cas9 recombinant vector;

(2) introducing the CRISPR/Cas9 recombinant vector in step (1) into potato cells, inducing the co-expression of the guide RNA expression cassette and the Cas9 nuclease expression cassette of the target fragment in the cell, cleaving the double-stranded target fragment of the S-RNase gene to trigger the DNA repair function of the potato cell itself, and causing random insertion or deletion of bases at the target site, thereby achieving a loss-of-function mutation of the intracellular S-RNase gene;

(3) screening for plants with a mutation in the S-RNase gene.

Another aspect of the present invention provides a method for breeding a potato, which comprises utilizing the potato plant, and a plant part, a tuber or tuber part, a plant cell, a pollen or a seed thereof described above, or the potato plant obtained by the above breeding method to perform self-crossing.

Another aspect of the present invention provides a method for manufacturing a commercial plant product, which comprises: obtaining the plant or a plant part thereof described above and manufacturing the commercial plant product from the plant or a plant part thereof, wherein the plant products are selected from the group consisting of: fresh whole potatoes, French fries, potato chips, dehydrated potato materials, potato flakes, and potato granules.

Another aspect of the present invention provides a food product made from a potato plant, a tuber, or a tuber part growing from the potato plant, and a plant part, a tuber or tuber part, a plant cell, a pollen or a seed thereof described above.

In a particular embodiment of the invention, the food product is a sliced potato tuber food.

In a particular embodiment of the invention, the food product is a group consisting of French fries, potato chips and baked potatoes.

The present invention adopts the above technical solutions, and it brings the following beneficial effects. Compared with the conventional methods for solving the self-incompatibility of potatoes, the present invention has the following advantages:

1) the invention performs directed editing of the self-incompatibility gene, constructing a vector which simultaneously targets the target sites of the two target genes, thereby creating a plurality of new self-compatible breeding materials;

2) the breeding period is short, and the entire process of the directed creation of the breeding material is about 12 months;

3) the artificial self-crossing is less affected by the flowering period and environment, saving time and labor, having strong maneuverability, higher fruit setting rate and more seeds, while self-crossing at the bud stage has large limitation, lower fruit setting rate and fewer seeds;

4) the self-compatibility of the self-compatible material created by the invention is heritable, and the barrier of self-incompatibility for diploid potatoes is fundamentally overcomed.

### DETAILED DESCRIPTION OF THE DRAWINGS

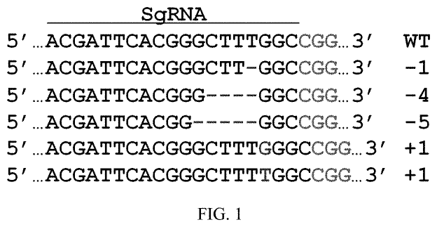

FIG. 1 shows the detection results of the target mutations according to an example of the present invention.

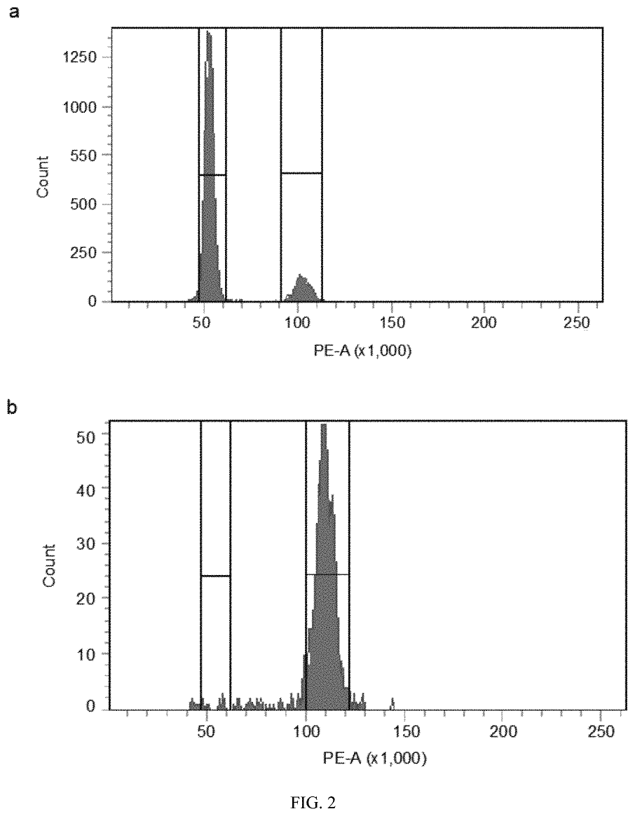

FIG. 2 shows the ploidy detection of the regenerated plants provided by an example of the present invention, wherein panel a is a diploid; panel b is a tetraploid.

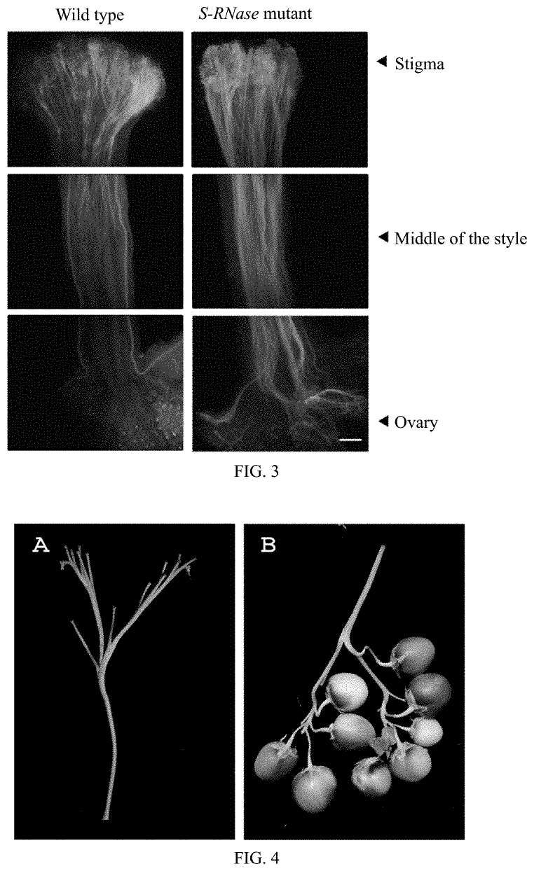

FIG. 3 is a phenotype diagram showing elongation of pollen tubes in the style of a wild type material (A) and a genetically edited material (B) according to an example of the present invention.

FIG. 4 is a phenotype diagram showing the fruit setting for self-crossing of a wild-type and a genetically edited plant line according to an example of the present invention.

### SPECIFIC EMBODIMENTS

The invention will be further described in detail below with reference to a particular example and the drawings.

### Example 1: The present invention provides a method for overcoming the

self-incompatibility of diploid potatoes, which comprises the following steps:

(1) selecting a target fragment in the first exon region of Sₚ₃ and Sₚ₄ in the S-RNase gene as a potato self-incompatibility determining gene; the target fragment in the step (1) is located on the target gene S-RNase, and one strand of the target fragment has the nucleic acid sequence structure as shown in SEQ ID No:1. One strand of the target fragment has the nucleic acid sequence structure as shown in SEQ ID No:1. For example, wherein the target fragment is located on the target S-RNase gene, one strand of the target fragment has a 5′-(N)_{X}-NGG-3′ structure, and (N)_{X} represents a base sequence having the base number of X {N₁, N₂, . . . , N_{X}}, and each of N₁, N₂, . . . N_{X} represents any one of bases A, G, C, and T, and N in NGG is any one of A, G, C, and T.

(2) constructing a CRISPR/Cas9 recombinant vector for diploid potato S-RNase gene-targeting according to the nucleic acid sequence of the target fragment obtained in step (1), wherein in the step (2) the recombinant vector comprises the target fragment, wherein the target fragment is the nucleic acid sequence of SEQ ID No:1 or a sequence complementary thereof;

The specific operations are as follows:

2.1 selecting a completely conservative nucleotide sequence ACGATTCACGGGCTTTGGCCGG on the first exon of the two S-RNase genes (Sₚ₃ and Sₚ₄) of the diploid potato S. phureja CIP 703541 (the last CGG part is the NGG portion in the 5′-(N)_{X}-NGG-3′ structure) as a targeting site. The nucleotide sequence of Sₚ₃ is shown as Seq ID No:2, the nucleotide sequence of Sₚ₄ is shown as Seq ID No:3; the target nucleotide sequence of Sₚ₃ is shown as positions 154 to 172 of Seq ID No:2, and the target nucleotide sequence of Sₚ₄ is shown as positions 157 to 175 of Seq ID No:3;

2.2 synthesizing the forward oligonucleotide strand (S-RNase P3) and the complementary reverse oligonucleotide strand (S-RNase P4) according to the selected target site.

The specific sequences are as follows:

S-RNase P3 (i.e., Seq ID No: 4):
xxxxACGATTCACGGGCTTTGGC,
S-RNase P4 (i.e., Seq ID No: 5):
xxxxGCCAAAGCCCGTGAATCGT;

wherein the portion not underlined is a sequence in above target site with deletion of NGG or a complementary sequence thereof, and the underlined portion is a cohesive end for ligation of the vector;

2.3 annealing the primers S-RNase P3 and S-RNase P4, and the two strands of S-RNase P3 and S-RNase P4 are annealed to form double-stranded DNA with cohesive ends as an insert fragment for constructing a recombinant vector;

2.4 digesting the pKSE401 vector with BsaI endonuclease at 50° C. for 12 hours, and inactivating the enzyme digestion system at 65° C. for 10 min, as a backbone fragment of the framework recombinant vector;

2.5 ligating the recombinant vector backbone fragment and the insert fragment by T4 ligase, then transferring into E. coli, after verification by sequencing, the positive transformants are extracted to form a recombinant vector plasmid for targeting the diploid potato S-RNase gene by CRISPR/Cas9;

2.6 transferring the recombinant vector plasmid into Agrobacterium EHA105 strain, and after sequencing, then extracting the positive transformed strain after verification by sequencing.

(3) introducing the recombinant vector obtained in the step (2) into potato cells, inducing the co-expression of the guide RNA expression cassette and the Cas9 nuclease expression cassette of the target fragment in the cell, cleaving the double-stranded target fragment of the S-RNase gene to trigger the DNA repair function of the potato cell itself, and causing random insertion or deletion of bases at the target site, thereby achieving a loss-of-function mutation of the intracellular S-RNase gene;

(4) regenerating a plurality of potato plants from the potato cells introduced with the recombinant vector, and screening the marker gene in the selected regeneration plants;

(5) specifically amplifying a DNA segment with the target fragment in the S-RNase gene of the selected regeneration plants by genomic PCR method, and sequencing the amplified products;

(6) selecting a regenerated plant in which the S-RNase gene is edited;

(7) detecting the ploidy of the selected gene-edited plant to select a diploid gene-edited plant line;

(8) propagating and planting the selected gene-edited plant line, and identifying the self-compatible phenotype at the flowering stage; and

(9) harvesting the seeds of the self-compatible plant line, extracting the genomic DNA of the offspring, and specifically amplifying a DNA segment with the target fragment in the S-RNase gene of the selected offspring by PCR method, then sequencing the amplified products and detecting the inheritance and isolation of the edited target gene in the offspring.

The specific operations for detecting the gene editing of the potato S-RNase in the above steps:

3.1 culturing the shoot tip of the aseptically preserved donor potato material S. phureja CIP 703541 on MS30 basal medium for 3 weeks, and taking the internodes as explants to plate on P-MS20 plate medium (2 pieces of sterile filter paper are previously placed on the surface of the medium, adding 2 mL of PACM solution) and pre-culturing for 2 days, the basic medium formula is as described in MS30, the pre-medium formula is as described in P-MS20, and the PACM solution is formulated as PACM;

3.2 activating positive transformed strain of Agrobacterium EHA105, shaking the bacteria to OD 0.5, then dipping and dyeing the pre-cultured explants described in the above step 2.1 for 15 minutes, then plating the explants on C-MS20 plate medium (1 piece of sterile filter paper is previously placed on the surface of the medium) and co-culturing in the dark for 2 days, and the common medium formulation is as described in C-MS20;

3.3 transferring the co-cultured explants from the end of step 3.2 onto D-MS20 plate differentiation medium for culturing, and the medium is changed every 14 days, and the differentiation medium formula is as described in D-MS20;

3.4 excising the extensible shoots produced by differentiation on the explants, and transferring onto the R-MS30 medium in the tissue culture flask for the resistance screening of the positive transformant, and the resistant screening medium formula is as described in R-MS30;

3.5 extracting the genomic DNA of the positive transformant as a template, and amplifying the full length of the two S-RNase genes Sₚa and Sₚ₄ by respectively using the specific primer pairs, Sₚ₃-F: GGGGAAACTGGAAAATGGTT (i.e., Seq ID No:6), Sₚ₃-R: ATGTGAAGTTGTTCAGCGAAA (i.e., Seq ID No:7), and SwF: CAACAAAATGGCTAAATCGCAG (i.e., Seq ID No:8), Sₚ₄-R: GGTTTTCTGTTGGGTGGCAT (i.e., Seq ID No:9); then detecting the target mutation of the target gene sequence by Sanger sequencing, and the results are shown in FIG. 1;

It can be seen from FIG. 1 that, in the present example five plants with target mutations are obtained, and all the S-RNase proteins undergo frameshift mutation.

3.6 detecting the positive transformants of the above target mutations by flow cytometry, and selecting the potato S-RNase gene-edited material, which still retains the diploid chromosomes. The detection results are shown in FIG. 2, wherein panel a is a diploid; panel b is a tetraploid. In the present example, five plants with target mutations are obtained, and their ploidy traits are all diploid types as shown in panel a.

4. Phenotypic identification of diploid potato S-RNase gene-edited material:

4.1 propagating and planting the diploid potato S-RNase gene-edited plant line, performing artificial self-crossing at the flowering stage;

4.2 48 hours after pollination, taking the pistil tissues of wild type and mutant plant lines respectively, fixing with 95% EtOH and glacial acetic acid in proportion of 3:1 for 24 hours, softening by 5M NaOH for 24 hours, and rinsing with ddH₂O, staining with 0.005 mg·mL⁻¹ aniline blue solution for 24 hours, and examining pollen tube dyeing under a fluorescence microscope. The detection results are shown in FIG. 3. According to the detection results, 48 hours after self-pollination, as for a group of wild-type materials S. phureja CIP 703541, the pollen tubes cannot enter the ovules, i.e., the wild type materials are self-incompatible; and 48 hours after self-pollination, as for the other group of donor material with orthomutation (S-RNase mutant), the pollen tubes successfully enter the ovules, indicating that the mutant plant line is self-compatible;

4.3 identifying the fruit setting phenotype of self-pollination for the wild-type and the mutant plant lines, and the detection results are shown in FIG. 4. FIG. 4A is a wild-type plant line, and FIG. 4B is a genetically edited plant line. It can be seen from FIG. 4 that, the wild type cannot bear fruits by self-crossing, and the mutant type can bear fruits by self-crossing, indicating that the mutant plant line is a self-compatible new material, and successfully overcomes the barrier of self-incompatibility;

4.4 harvesting the seeds of the self-compatible plant line, and sowing the seeds in the aperture disk; after the true leaves come out, extracting the genomic DNA of all the seedlings; then specifically amplifying the two DNA segments containing the target fragments in the S-RNase gene of the selected seedlings by PCR, and sequencing the amplified products to detect the inheritance and isolation of the edited target gene in the offspring. It is verified that the self-compatibility of the new material created by site-directed gene editing of the S-RNase can be passed on to the offspring. The verification results are shown in Table 1.

TABLE 1
Mutation patterns of T0 and T1 generation plant lines of the gene-edited materials

| | T₀ generation | | T₁ generation | | | | | |
|---|---|---|---|---|---|---|---|---|
| No. | Sp3 | Sp4 | Cas9-free^{a} | Sp3Sp3^{b} | Sp3Sp4 | | Sp4Sp4 | |
| 32 | chimeric | +1 bp | 7/192 | 0 | 3 | (Sp4)^{c} | 4 | (Sp4) |
| 42 | wild type | −5 bp | 45/192 | 0 | 17 | (Sp4) | 28 | (Sp4) |

| | | | | | | | | |
|---|---|---|---|---|---|---|---|---|
| 44 | −4 bp | chimeric | 47/192 | 20 | (Sp3) | 27 | (Sp3) | 0 |

| | | | | | | | | |
|---|---|---|---|---|---|---|---|---|
| 57 | wild type | chimeric | 13/136 | 0 | 6 | (Sp4) | 7 | (Sp4) |

| | | | | | | | | |
|---|---|---|---|---|---|---|---|---|
| 66 | −1 bp | wild type | 27/192 | 14 | (Sp3) | 13 | (Sp3) | 0 |

Note:
^{a}The number before the slash represents the number of individual plants without Cas9 in the detected T₁ generation, the number after the slash represents the number of individual plants in the detected T₁ generation;
^{b}the isolation of S-RNase type for the individual plants without Cas9 in the T₁ generation;
^{c}indicates the S-RNase mutation type.

The medium formulations used above are shown in the following tables: MS30 (1 L):

| MS | 4.43 | g |
|---|---|---|
| sucrose | 30 | g |
| pH | 5.8 | |
| agar | 8 | g |

### P-MS20 (1 L):

| MS | 4.43 | g |
|---|---|---|
| sucrose | 20 | g |
| pH | 5.8 | |
| agar | 8 | g |

### PA-MS20 (1 L):

| | | |
|---|---|---|
| MS | 4.43 | g |
| sucrose | 20 | g |
| caseine hydrolysate | 2 | g |
| 2,4-D | 1 | mg/L |
| KT | 0.5 | mg/L |

| | |
|---|---|
| pH | 6.5 |

### C-MS20 (1 L):

| MS | 4.43 | g |
|---|---|---|
| sucrose | 20 | g |
| pH | 5.8 | |
| agar | 8 | g |
| a-napthaleneacetic acid | 2 | mg · L⁻¹ |
| trans-zeatin | 1 | mg · L⁻¹ |
| AS | 40 | mg · L⁻¹ |

### D-MS20 (1 L):

| MS | 4.43 | g |
|---|---|---|
| sucrose | 20 | g |
| pH | 5.8 | |
| agar | 8 | g |
| a-napthaleneacetic acid | 0.01 | mg · L⁻¹ |
| trans-zeatin | 2 | mg · L⁻¹ |
| kanamycin | 100 | mg · L⁻¹ |
| temetine | 200 | mg · L⁻¹ |

### R-MS30 (1 L):

| MS | 4.43 | g |
|---|---|---|
| sucrose | 30 | g |
| pH | 5.8 | |
| agar | 8 | g |
| kanamycin | 50 | mg · L⁻¹ |
| temetine | 200 | mg · L⁻¹ |

The above description is only the preferred example of the present invention, and is not intended to limit the present invention. For those skilled in the art, various modifications and changes can be made to the present invention. Any modifications, equivalent substitutions, improvements, etc. made within the spirit and the scope of the present invention should be included in the scope of the present invention.

1. A method for overcoming self-incompatibility of diploid potatoes, comprising the following steps:
(1) selecting a target fragment in the gene regions of Sₚa and Sₚ₄ in the S-RNase gene as a potato self-incompatibility determining gene;
(2) constructing a CRISPR/Cas9 recombinant vector for diploid potato S-RNase gene-targeting according to the nucleic acid sequence of the target fragment obtained in step (1);
(3) introducing the recombinant vector obtained in the step (2) into potato cells, inducing the co-expression of the guide RNA expression cassette of the target fragment and the Cas9 nuclease expression cassette in the cell, cleaving the double-stranded target fragment of the S-RNase gene to trigger the DNA repair function of the potato cell itself, and causing random insertion or deletion of bases at the target site, thereby achieving a loss-of-function mutation of the intracellular S-RNase gene;
(4) regenerating a plurality of potato plants from the potato cells introduced with the recombinant vector, and screening the marker gene in the selected regeneration plants;
(5) specifically amplifying a DNA segment with the target fragment in the S-RNase gene of the selected regeneration plants by genomic PCR method, and sequencing the amplified products;
(6) selecting a regenerated plant in which the S-RNase gene is edited;
(7) detecting the ploidy of the selected gene-edited plant to select a diploid gene-edited plant line;
(8) propagating and planting the selected gene-edited plant line, and identifying the self-compatible phenotype at the flowering stage; and
(9) harvesting the seeds of the self-compatible plant line, extracting the genomic DNA of the offspring, and specifically amplifying a DNA segment with the target fragment in the S-RNase gene of the selected offspring by PCR method, then sequencing the amplified products and detecting the inheritance and isolation of the edited target gene in the offspring.

2. The method for overcoming self-incompatibility of diploid potatoes according to claim 1, characterized in that in the step (1) the target fragment is located on the target gene S-RNase, and one strand of the target fragment has the nucleic acid sequence structure as shown in SEQ ID No:1.

3. The method for overcoming self-incompatibility of diploid potato according to claim 2, wherein in the step (2) the recombinant vector comprises the target fragment, wherein the target fragment is the nucleic acid sequence of SEQ ID No:1 or a sequence complementary thereof.

4. A potato plant, and a plant part, a tuber or tuber part, a plant cell, a pollen or a seed thereof, wherein it comprises a loss-of-function mutation of the S-RNase gene, wherein the nucleotide sequence of the S-RNase gene is the sequence shown in SEQ ID NO:2 (Sₚ₃), or a complementary sequence, a degenerate sequence, or a homologous sequence thereof; and/or the sequence shown in SEQ ID NO:3 (Sₚ₄), or a complement sequence, a degenerate sequence, or a homologous sequence thereof.

5. The potato plant, and a plant part, a tuber or tuber part, a plant cell, a pollen or a seed thereof according to claim 4, wherein the homologous sequence has a homology of 30% or more, 40% or more, 50% or more, 60% or more, 70% or more, 80% or more, 90% or more, 95% or more, 96% or more, 97% or more, 98% or more, 99% or more, 99.5% or more, or 99.9% or more.

6. The potato plant, and a plant part, a tuber or tuber part, a plant cell, a pollen or a seed thereof according to claim 4, wherein the loss-of-function mutation of the S-RNase gene is achieved by addition and/or deletion of (one or more) nucleotides in the gene expressing the S-RNase protein.

7. The potato plant, and a plant part, a tuber or tuber part, a plant cell, a pollen or a seed thereof according to claim 6, wherein it is achieved by addition, deletion or replacement of (one or more) nucleotides in the sequence of 5′-(N)_{X}-NGG-3′ structure or a complementary sequence thereof.

8. The potato plant, and a plant part, a tuber or tuber part, a plant cell, a pollen or a seed thereof according to claim 7, wherein it is achieved by addition, deletion or replacement of (one or more) nucleotides in the sequence of ACGATTCACGGGCTTTGGCC or a complementary sequence thereof.

9. The potato plant, and a plant part, a tuber or tuber part, a plant cell, a pollen or a seed thereof according to claim 6, wherein the addition and/or deletion of nucleotides is achieved by a CRISPR/Cas9 recombinant vector.

10. The potato plant, and a plant part, a tuber or tuber part, a plant cell, a pollen or a seed thereof according to claim 9, wherein the nucleotide sequence of the sgRNA in the CRISPR/Cas9 recombinant vector is:
S-RNase P3 (i.e., Seq ID No:4): xxxxACGATTCACGGGCTTTGGC,
S-RNase P4 (i.e., Seq ID No:5): xxxxGCCAAAGCCCGTGAATCGT;
wherein the portion not underlined is a sequence in above target site with deletion of NGG or a complementary sequence thereof, and the underlined portion is a cohesive end for ligation of the vector.

11. A CRISPR/Cas9 recombinant vector for targeted knockout of S-RNase protein gene, wherein the nucleotide sequence of the S-RNase protein targeted by the CRISPR/Cas9 recombinant vector is the sequence shown in SEQ ID NO:2 (Sₚ₃), or a complementary sequence, a degenerate sequence, or a homologous sequence thereof; and/or the sequence shown in SEQ ID NO:3 (Sₚ₄), or a complement sequence, a degenerate sequence, or a homologous sequence thereof.

12. The recombinant vector according to claim 11, wherein the nucleotide sequence of the sgRNA in the CRISPR/Cas9 recombinant vector is:
S-RNase P3 (i.e., Seq ID No:4): xxxxACGATTCACGGGCTTTGGC,
S-RNase P4 (i.e., Seq ID No:5): xxxxGCCAAAGCCCGTGAATCGT;
wherein the portion not underlined is a sequence in above target site with deletion of NGG or a complementary sequence thereof, and the underlined portion is a cohesive end for ligation of the vector.

13. Use of the CRISPR/Cas9 recombinant vector of claim 11 in the preparation of the knockout of S-RNase protein gene.

14. A method for breeding a self-compatible potato, comprising the step of making the S-RNase gene in a potato unexpressed or inactivated, wherein the S-RNase gene is the sequence shown in SEQ ID NO:2 (Sₚ₃), or a complementary sequence, a degenerate sequence, or a homologous sequence thereof; and/or the sequence shown in SEQ ID NO:3 (Sₚ₄), or a complement sequence, a degenerate sequence, or a homologous sequence thereof.

15. A method for breeding a potato, comprising utilizing the potato plant, and a plant part, a tuber or tuber part, a plant cell, a pollen or a seed thereof according to claim 4 to perform self-crossing.

16. (canceled)

17. (canceled)

18. A method for breeding a potato, comprising the potato plant obtained by the breeding method according to claim 14 to perform self-crossing.

19. A method for manufacturing a commercial plant product, which comprises: obtaining the plant or a plant part thereof according to claim 4 and manufacturing the commercial plant product from the plant or a plant part thereof, wherein the plant products are selected from the group consisting of: fresh whole potatoes, French fries, potato chips, dehydrated potato materials, potato flakes, and potato granules.

20. A food product made from a potato plant, a tuber, or a tuber part growing from the potato plant, and a plant part, a tuber or tuber part, a plant cell, a pollen or a seed thereof according to claim 4.
